(19) United States
(12) Patent Application Publication
(10) Pub. No.: US 2023/0201234 A1
(43) Pub. Date: Jun. 29, 2023

(54) FAST DISPERSIBLE PHARMACEUTICAL COMPOSITION COMPRISING CAPECITABINE

(71) Applicant: SHILPA MEDICARE LIMITED, RAICHUR (IN)

(72) Inventor: NIJAGUNI REVANSIDDAYYA RUDRASWAMY MATH, Bengaluru (IN)

(73) Assignee: SHILPA MEDICARE LTD, RAICHUR (IN)

(21) Appl. No.: 17/927,004

(22) PCT Filed: May 29, 2021
(86) PCT No.: PCT/IB2021/054728

§ 371 Date: Nov. 22, 2022

(30) Foreign Application Priority Data
Jun. 1, 2020 (IN) 202041022831

(51) Int. Cl. A61K 31/7068; A61K 9/20

(52) U.S. Cl. CPC A61K 31/7068; A61K 9/2054; A61K 9/2009; A61K 9/2027; A61K 9/2013

(57) ABSTRACT

The present invention relates to a pharmaceutical composition, preferably in form of tablet(s), in particular a dispersible, especially a fast dispersible pharmaceutical composition, preferably in the form of tablet(s), further in particular a water dispersible, especially a fast water-dispersible pharmaceutical composition, preferably in form of tablet(s), comprising capecitabine with at least one pharmaceutically acceptable excipient, preferably the pharmaceutical composition being intended to be dispersed in water and/or other aqueous liquids before oral administration; in particular, this pharmaceutical composition can be characterized with disintegration time of less than 3 minutes using purified water at 15° C. to 25° C. (Ph. Eur Disintegration Test).

### FIELD OF THE INVENTION

The present invention generally relates to pharmaceutical composition comprising capecitabine or a pharmaceutically acceptable salts thereof together with at least one pharmaceutically acceptable excipient intended to be dispersed in water before oral administration. The invention also refers to the process for the preparation of said pharmaceutical preparations with fast disintegration in water or other aqueous liquids such as fruit juices where fast disintegration means that said pharmaceutical composition disintegrates in less than 3 minutes using purified water at 15° C. to 25° C. (Ph. Eur Disintegration test).

### BACKGROUND OF THE INVENTION

Capecitabine is a fluoropyrimidine carbamate with antineoplastic activity. It is an orally administered systemic prodrug of 5′-deoxy-5-fluorouridine (5′-DFUR), an antineoplastic agent. Capecitabine is marketed in the United States by Roche Laboratories under the brand name Xeloda®. The chemical name for capecitabine is 5′-deoxy-5-fluoro-N-[(pentyloxy) carbonyl]-cytidine and has the following structural formula

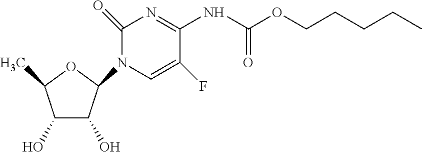 [SMILES: CCCCCOC(=O)Nc1nc(=O)n([C@@H]2O[C@H](C)[C@@H](O)[C@H]2O)cc1F]

Capecitabine is currently approved in United States for the treatment of Colon Cancer, Colorectal Cancer and Breast Cancer. The recommended dose of capecitabine in these indications is 1250 mg/m2 administered orally twice daily (equivalent to 2500 mg/m2 total daily dose) for 14 days followed by a 7-day rest period given as three-week cycles, for as long as needed. Typically, the mean duration of treatment is three to 6-week cycles. The currently approved unit dosage forms are film coated tablets containing 150 mg and 500 mg of capecitabine.

These currently approved film coated tablets of capecitabine may be difficult to swallow by the pediatric and geriatric populations, as well as by patients with swallowing impediments and blockages.

The capecitabine tablet currently on the market (Xeloda®) typically requires approximately 7-12 minutes to disintegrate in water (USP Disintegration Test), depending on the size of the tablet. Traditional excipients currently used in these tablets, such as lactose and croscarmellose sodium, by themselves do not overcome the cohesive property of capecitabine in the tablet. The end result is that the marketed tablet slowly disintegrates by surface erosion and is thus not very amenable to rapid dispersion or disintegration in water prior to oral administration to swallowing compromised patients.

PCT Publication No. WO 2008/040665 discloses the film coated pharmaceutical composition comprising capecitabine and at least one disintegrant, said composition disintegrating in water at 37° C. in a USP Disintegrating Apparatus in less than 2 minutes and having a hardness of 8-13 SCU.

PCT Publication No. WO 2010/069795 discloses the film coated pharmaceutical composition comprising capecitabine and at least one disintegrant consisting of formulation of mannitol (90%), crospovidone (5%) and polyvinyl acetate (5%), said composition being characterized by disintegrating in water at 37° C. in a USP Disintegration Apparatus in less than 2.5 minutes and having a hardness of 8-23 SCU.

CN102988320B discloses a preparation method for capecitabine dispersible tablet comprising 80% capecitabine, 7.5% microcrystalline cellulose, 7.5% sodium carboxymethyl cellulose and 5% polyvinyl pyrrolidone.

There is a constant need for development of pharmaceutical formulations of capecitabine which would be most convenient for patients and would offer advantages over traditional dosage forms. Considering the prior art efforts related to capecitabine as disclosed above, a need for formulations intended to be dispersed in water or fruit juice still exists. Therefore, there exists a need to develop the pharmaceutical composition comprising capecitabine or a pharmaceutically acceptable salts thereof together with at least one pharmaceutically acceptable excipient intended to be dispersed in water or fruit juices before oral administration.

### SUMMARY OF THE INVENTION

According to one aspect of the present invention, the present invention provides a pharmaceutical composition, preferably in form of tablet(s), in particular a dispersible, especially a fast dispersible pharmaceutical composition, preferably in the form of tablet(s), further in particular a water dispersible, especially a fast water-dispersible pharmaceutical composition, preferably in form of tablet(s), comprising capecitabine with at least one pharmaceutically acceptable excipient, preferably the pharmaceutical composition being intended to be dispersed in water and/or other aqueous liquids before oral administration; in particular, this pharmaceutical composition can be characterized with disintegration time of less than 3 minutes using purified water using purified water at 15° C. to 25° C. (Ph. Eur USP Disintegration test).

In another aspect of the present invention, the present invention provides a fast water dispersible tablet composition comprising of about 50% w/w to about 85% w/w of capecitabine, about 10% w/w to about 25% w/w of diluents, about 1% w/w to about 5% w/w of disintegrants, about 1% w/w to about 5% w/w of glidant and about 0.1% w/w to about 2% w/w of lubricants.

In a still further aspect of the present invention, the present invention provides a fast water dispersible tablet composition comprising of about 50% w/w to about 85% w/w of capecitabine, about 10% w/w to about 25% w/w of diluents, about 1% w/w to about 5% w/w of disintegrants, about 1% w/w to about 5% w/w of glidant, about 0.1% w/w to about 2% w/w of lubricants, about 0.5% w/w to about 5% w/w of sweetening agents and about 0.1% w/w to about 1.5% w/w of flavoring agents.

### DETAILED DESCRIPTION OF THE INVENTION

The present invention relates to the rapidly disintegrating pharmaceutical composition comprising capecitabine or pharmaceutically acceptable salts thereof in the form of tablets.

Capecitabine used as active ingredient in the pharmaceutical composition according to present invention refers to capecitabine as well as pharmaceutically acceptable salts, enantiomers, racemates, solvates, hydrates, amorphous and crystal forms or combinations thereof.

The present invention further provides a pharmaceutical composition, preferably in form of tablet(s), in particular a dispersible, especially a fast dispersible pharmaceutical composition, preferably in the form of tablet(s), further in particular a water dispersible, especially a fast water-dispersible pharmaceutical composition, preferably in form of tablet(s), comprising capecitabine with at least one pharmaceutically acceptable excipient, preferably the pharmaceutical composition being intended to be dispersed in water and/or other aqueous liquids before oral administration; in particular, this pharmaceutical composition can be characterized with disintegration time of less than 3 minutes using purified water at using purified water at 15° C. to 25° C. (Ph. Eur Disintegration test).

Capecitabine mostly used in the present invention is capecitabine base. The amount of capecitabine present in the pharmaceutical composition shall be of about 50% w/w to about 85% w/w by weight of the pharmaceutical composition, more preferably about 60% w/w to about 80% w/w and most preferably about 65% w/w to about 75% w/w of the pharmaceutical composition (based on the total weight of the composition). In particular, the pharmaceutical composition (which can be dispersible, in particular water-dispersible, especially fast dispersible, in particular fast water-dispersible) can be in the form of tablet(s) comprising capecitabine and further comprising pharmaceutically acceptable excipients selected from the group consisting of diluents, disintegrants, glidants, lubricants, sweetening agents and flavoring agents.

A tablet comprising capecitabine (which can be a dispersible, in particular water-dispersible, especially fast dispersible, in particular fast water-dispersible tablet) can comprise a mixture, preferably compressed mixture, comprising
a) granulate, in particular granulate prepared by granulation, in particular dry granulation (slugging or roll compaction), said granulate comprising capecitabine, and
b) a pharmaceutically acceptable excipient or mixture comprising or consisting of two or more pharmaceutically acceptable excipients (which pharmaceutically acceptable excipients shall be referred herein as extragranular excipients and the pharmaceutically acceptable excipient or mixture comprising or consisting of two or more further pharmaceutically acceptable excipients can be also referred to herein as extragranular phase).

According to one embodiment, the terms “granulate” and “granule(s)” can have the same meaning.

The granulate may comprise one or more further pharmaceutically acceptable excipients, these one or more further pharmaceutically acceptable excipients can be also referred to herein as intragranular excipients. Excipients present in the granulate shall be referred to herein as intragranular used excipients. The granulate comprising capecitabine present in the pharmaceutical composition, especially tablet, can be also referred to herein as intragranular phase.

In one embodiment, a tablet comprising capecitabine (especially a dispersible, in particular water-dispersible, especially fast dispersible, in particular fast water dispersible tablet comprising capecitabine) can comprise pharmaceutically acceptable excipients selected from group consisting of diluents, disintegrants, glidants, lubricants, sweetening agents and flavoring agents.

In one embodiment, the present invention provides a fast water dispersible tablet composition comprising of about 50% w/w to about 85% w/w of capecitabine, about 10% w/w to about 25% w/w of diluents, about 1% w/w to about 5% w/w of disintegrants, about 1% w/w to about 5% w/w of glidant and about 0.1% w/w to about 2% w/w of lubricants.

In a further embodiment, the present invention provides a fast water dispersible composition comprising of about 50% w/w to about 85% w/w of capecitabine, about 10% w/w to about 25% w/w of diluents, about 1% w/w to about 5% w/w of disintegrants, about 1% w/w to about 5% w/w of glidant, about 0.1% w/w to about 2% w/w of lubricants, about 0.5% w/w to about 5% w/w of sweetening agents and about 0.1% w/w to about 1.5% w/w of flavoring agents.

The pharmaceutical composition, preferably in the form of tablet(s), of the present invention can be obtained according to any procedure disclosed in the present application especially by a process comprising the steps:
i. production of capecitabine granulate,
ii. blending of capecitabine granulate with extragranular excipients,
iii. blending a lubricant with a mixture obtained in step ii) to obtain final compression mixture,
iv. compressing of the compression mixture obtained in step iii) into tablets.

The term “fast dispersible tablets” as used herein means tablets intended to be dispersed in water and/or other aqueous liquids such as fruit juices, preferably apple juice, before administration, giving a dispersion, preferably homogenous dispersion. Fast dispersible tablets according to present invention can disintegrate in less than 3 minutes using purified water at using purified water at 15° C. to 25° C. (Ph. Eur Disintegration test). “Dispersible tablets”, “pharmaceutical composition”, “pharmaceutical preparation”, “pharmaceutical formulation”, “composition”, “formulation”, “medicine”, “rapidly disintegrating tablets”, “solid medicinal preparation”, “final dosage form”, “final product”, “tablet dosage form”, “final composition”, “tablets” are all terms used in the present invention which equally characterize “fast dispersible tablets” comprising capecitabine together with at least one pharmaceutically acceptable excipient intended to be dispersed in water and/or other aqueous liquids before oral administration.

Diluents used in the present invention are selected from starch and its derivates, such as corn starch, pregelatinized starch, and dextrins, cellulose and its derivatives, such as microcrystalline cellulose or co-processed microcrystalline cellulose such as silicified microcrystalline cellulose, carbohydrates and its derivatives, in particular simple carbohydrates or their derivatives, such as glucose, lactose, and sucrose, sugar alcohols, such as mannitol, xylitol and sorbitol, metal salts of phosphoric acid, such as calcium hydrogen phosphate in anhydrous or hydrated form, or other diluents and combinations thereof not specifically listed herein. Diluent is preferably present in the tablet in an amount of about 10% w/w to about 25% w/w based on the total weight of the tablet dosage form. Particularly the preferred diluent is microcrystalline cellulose. In the present invention, the whole of diluent is included in the intragranular portion.

Disintegrants used in the present invention are selected from crospovidone, croscarmellose sodium, sodium starch glycolate and low substituted hydroxypropyl cellulose. Preferably crospovidone is used as a disintegrant in the present invention. Disintegrant is preferably present in the tablet in an amount of about 1% w/w to about 5% w/w based on the total weight of the tablet dosage form. In the present invention, the whole of disintegrant is included in the intragranular portion.

Glidants used in the present invention are selected from talc, fumed silica (colloidal silicon dioxide), magnesium oxide, silicates such as magnesium silicate, polyethylene glycols or other glidants or combinations thereof not specifically listed herein. The most preferably used glidant in the present invention is colloidal silicon dioxide. Preferred amount of the glidant in the composition of the present invention is in the range of about 1% w/w to about 5% w/w based on the total weight of the tablet dosage form. In the foregoing embodiments, the glidant can be included intragranular in an amount of at least 1% w/w based on the total weight of the tablet dosage form. In the foregoing embodiments, the glidant can be included in both the intragranular and extragranular portion. It has been found that inclusion of glidant within granule in an amount of at least 1% w/w based on the total weight of the tablet dosage form advantageously improves disintegration time, hardness and dissolution properties of the tablet dosage form. Glidant is present in the intragranular portion in an amount of about 1% w/w to about 2% w/w based on the total fast dispersible tablet dosage form. Glidant is present in the extragranular portion in an amount of about 1% w/w to about 2% w/w based on the total fast dispersible tablet dosage form. More preferably, colloidal silicon dioxide as a glidant is present in both intragranular and extragranular portions. The amount of glidant present in intragranular portion shall be about 20% w/w to 80% w/w of the total glidant quantity in the formulation. Further, the amount of glidant present in extragranular portion shall be about 20% w/w to 80% w/w of the total glidant quantity in the formulation.

Lubricants used in the present invention are sodium stearyl fumarate, sodium lauryl sulfate, magnesium stearate, glyceryl behenate. Most preferably used lubricant is magnesium stearate. Preferred amount of the lubricant in the composition of the present invention is in the range of about 0.1% w/w to about 2% w/w based on the total weight of the tablet dosage form. Lubricant is included in both the intragranular and extragranular portion.

Sweetening agents used in the present invention are selected from aspartame, sucralose, dextrose, fructose, ammonium glycyrrhizinate, maltose, mannitol, sorbitol and xylitol and/or combinations thereof. Sweeting agents used in the present invention is in the range of about 0.5% w/w to about 5% w/w based on the total weight of the tablet dosage form.

Flavoring agents used in the present invention are selected from Examples of the flavour agents are selected from the group consisting of peppermint flavour, cooling flavour (menthol), flavour oils, flavouring aromatic oil, peppermint oil, clove oil, bay oil, anise oil, eucalyptus oil thyme oil, oil of bitter almonds. Flavouring agents include, vanilla, chocolate flavour, citrus oils, fruit essences like Orange, strawberry, banana, and any combinations thereof. Flavoring agents used in the present invention is in the range of about 0.1% w/w to about 1.5% w/w based on the total weight of the tablet dosage form.

In one embodiment of the invention, the present invention provides a fast water dispersible tablet composition comprising
(a) about 50% w/w to about 85% w/w capecitabine,
(b) about 10% w/w to about 25% w/w microcrystalline cellulose,
(c) about 1% w/w to about 5% w/w crospovidone,
(d) about 1% w/w to about 5% w/w colloidal silicon dioxide and
(e) about 0.1% w/w to about 2% w/w magnesium stearate.

In another embodiment of the invention the present invention provides a fast water dispersible tablet composition comprising
(a) about 50% w/w to about 85% w/w capecitabine,
(b) about 10% w/w to about 25% w/w microcrystalline cellulose,
(c) about 1% w/w to about 5% w/w crospovidone,
(d) about 1% w/w to about 5% w/w colloidal silicon dioxide and
   (f) about 0.1% w/w to about 2% w/w magnesium stearate,
      wherein half the amount of colloidal silicon dioxide is intragranular and half the amount of colloidal silicon dioxide is extragranular.

In another embodiment of the invention the present invention provides a fast water dispersible tablet composition comprising
(a) about 50% w/w to about 85% w/w capecitabine,
(b) about 10% w/w to about 25% w/w microcrystalline cellulose,
(c) about 1% w/w to about 5% w/w crospovidone,
(d) about 1% w/w to about 5% w/w colloidal silicon dioxide and
(e) about 0.1% w/w to about 2% w/w magnesium stearate,
   wherein tablet comprises about 1% w/w to about 2% w/w of intragranular colloidal silicon dioxide and about 1% w/w to about 2% w/w of extragranular colloidal silicon dioxide based on the total weight of the tablet.

In a specific embodiment of the invention the present invention provides a fast water dispersible tablet composition comprising
(a) about 73% w/w of capecitabine,
(b) about 2.2% w/w crospovidone
(c) about 18.8% w/w microcrystalline cellulose,
(d) about 3% w/w colloidal silicon dioxide,
(e) about 1% w/w of magnesium stearate.

In another embodiment of the invention the present invention provides a fast water dispersible tablet composition comprising
(a) about 73% w/w of capecitabine,
(b) about 2.2% w/w crospovidone
(c) about 18.8% w/w microcrystalline cellulose,
(d) about 3% w/w colloidal silicon dioxide,
(e) about 1% w/w of magnesium stearate.
   wherein half the amount of colloidal silicon dioxide is intragranular and half the amount of colloidal silicon dioxide is extragranular.

In one embodiment of the invention the present invention relates to a fast water dispersible tablet comprising
(i) intragranular components:

| | | |
|---|---|---|
| Capecitabine | 1000 | mg |
| Crospovidone | 30 | mg |
| Colloidal silicon dioxide | 20 | mg |
| Microcrystalline cellulose | 257 | mg |
| Magnesium Stearate | 3.25 | mg |

and
(ii) extragranular components:

| | | |
|---|---|---|
| Colloidal silicon dioxide | 20 | mg |
| Magnesium Stearate | 9.75 | mg |

In a further embodiment of the invention the present invention relates to a fast water dispersible tablet consisting essentially of
(i) intragranular components:

| | | |
|---|---|---|
| Capecitabine | 1000 | mg |
| Crospovidone | 30 | mg |
| Colloidal silicon dioxide | 20 | mg |
| Microcrystalline cellulose | 257 | mg |
| Magnesium Stearate | 3.25 | mg |

and
(ii) extragranular components:

| | | |
|---|---|---|
| Colloidal silicon dioxide | 20 | mg |
| Magnesium Stearate | 9.75 | mg |

In another embodiment of the invention the present invention relates to a fast water dispersible tablet consisting of
(i) intragranular components:

| | | |
|---|---|---|
| Capecitabine | 1000 | mg |
| Crospovidone | 30 | mg |
| Colloidal silicon dioxide | 20 | mg |
| Microcrystalline cellulose | 257 | mg |
| Magnesium Stearate | 3.25 | mg |

and
(ii) extragranular components:

| | | |
|---|---|---|
| Colloidal silicon dioxide | 20 | mg |
| Orange or Strawberry flavor | 10 | mg |
| Aspartame | 20 | mg |
| Magnesium Stearate | 9.75 | mg |

The present inventors have found that the fast disintegration property of less than 3 minutes of the present tablets is achieved if the formulation is prepared without using a water-soluble binder.

The present inventors have found that the disintegrating time is less than 3 minutes if the tablet hardness does not exceed certain value. Hardness of the tablet is preferably between 100 N and 200 N. Hardness is tested using Electrolab EH-01P hardness tester, every time 10 tablets were tested and average tablet hardness is calculated.

The following examples are provided to illustrate the present invention. It is understood, however, that the invention is not limited to the specific conditions or details described in the examples below. The examples should not be construed as limiting the invention as the examples merely provide specific methodology useful in the understanding and practice of the invention and its various aspects. While certain preferred and alternative embodiments of the invention have been set forth for purposes of disclosing the invention, modification to the disclosed embodiments can occur to those who are skilled in the art.

### EXAMPLES

### Example 1: Fast Dispersible Tablets of Capecitabine

Composition

| | | | |
|---|---|---|---|
| S. No | Ingredients | Qty/Tablet (mg) | % w/w |

Intragranular Portion

| | | | |
|---|---|---|---|
| 1. | Capecitabine | 1000 | 72.99 |
| 2. | Crospovidone | 30 | 2.19 |
| 3. | Colloidal silicon dioxide | 20 | 1.46 |
| 4. | Microcrystalline cellulose | 257 | 18.76 |
| 5. | Magnesium Stearate | 3.25 | 0.24 |

Extragranular Portion

| | | | |
|---|---|---|---|
| 6. | Colloidal silicon dioxide | 20 | 1.46 |
| 7. | Orange/Strawberry flavor | 10 | 0.73 |
| 8. | Aspartame | 20 | 1.46 |
| 9. | Magnesium Stearate | 9.75 | 0.71 |

| | |
|---|---|
| Total Tablet weight | 1370 |

Capecitabine, intragranular portion excipients crospovidone, colloidal silicon dioxide, microcrystalline cellulose and magnesium stearate are mixed and blended up to 20 minutes and compacted by roll compacter. The compacts or are passed through the oscillating granulator and collected in blender. The extragranular portion excipients of colloidal silicon dioxide, Orange/Strawberry flavor and Aspartame are mixed with the blended intragranular portion and mixed in the blender. Further the magnesium stearate of the extragranular portion is added and mixed to form the final compression mixture, which was compressed into tablets.

### Comparative Example 1: Formulation without Intragranular Glidant

Composition

| | | |
|---|---|---|
| S. No | Ingredients | Qty/Tablet (mg) |

Intragranular Portion

| | | |
|---|---|---|
| 1. | Capecitabine | 1000 |
| 2. | Crospovidone | 30 |
| 3. | Microcrystalline cellulose | 257 |
| 4. | Magnesium Stearate | 3.25 |

Extragranular Portion

| | | |
|---|---|---|
| 5. | Colloidal silicon dioxide | 20 |
| 6. | Orange/Strawberry flavor | 10 |
| 7. | Aspartame | 20 |
| 8. | Magnesium Stearate | 9.75 |

| | |
|---|---|
| Total Tablet weight | 1350 |

The tablets were manufactured as the process disclosed in example 1 without the use of intragranular colloidal silicon dioxide.

### Example 2: Hardness Testing

(iii) Hardness of the tablets of example 1 and comparative example 1 are tested with Electrolab EH-01P hardness tester, every time 10 tablets of example 1 and comparative example-1 were tested and average tablet hardness is calculated as below

| Example | Example 1 | Comparative Example 1 |
|---|---|---|
| Hardness of Tablet | 148N to 152N | 98N to 102N |

### Example 3: Disintegration Testing

Disintegrating testing of tablets of example 1 and comparative example 1 are carried out in using purified water at 15° C. to 25° C. (Ph. Eur Disintegration test).

| Example | Example 1 | Comparative Example 1 |
|---|---|---|
| Disintegration Time | 1 minute 12 Seconds | 4 minutes 50 Seconds |

It was observed that the example 1 having the intragranular colloidal silicon dioxide (glidant) has the less disintegration time.

We claim:

1. A fast water dispersible tablet composition comprising
(a) about 50% w/w to about 85% w/w capecitabine,
(b) about 10% w/w to about 25% w/w diluents,
(c) about 1% w/w to about 5% w/w disintegrants,
(d) about 1% w/w to about 5% w/w glidant and
(e) about 0.1% w/w to about 2% w/w lubricants.

2. The fast water dispersible tablet as claimed in claim 1, wherein diluents are selected from group consisting of microcrystalline cellulose and silicified microcrystalline cellulose.

3. The fast water dispersible tablet as claimed in claim 1, wherein disintegrants are selected from group consisting of crospovidone, croscarmellose sodium, sodium starch glycolate and low substituted hydroxypropyl cellulose.

4. The fast water dispersible tablet as claimed in claim 1, wherein glidants are selected from group consisting of talc and colloidal silicon dioxide.

5. The fast water dispersible tablet as claimed in claim 1, wherein lubricants are selected from the group consisting of odium stearyl fumarate, sodium lauryl sulfate, magnesium stearate and glyceryl behenate.

6. A fast water dispersible tablet composition comprising
(a) about 50% w/w to about 85% w/w capecitabine,
(b) about 10% w/w to about 25% w/w microcrystalline cellulose,
(c) about 1% w/w to about 5% w/w crospovidone,
(d) about 1% w/w to about 5% w/w colloidal silicon dioxide and
(e) about 0.1% w/w to about 2% w/w magnesium stearate.

7. The fast water dispersible tablet as claimed in claim 6, wherein colloidal silicon dioxide is present in both intragranular and extragranular portion of the tablet.

8. The fast water dispersible tablet as claimed in claim 7, wherein the tablet comprises about 1% w/w to about 2% w/w of intragranular colloidal silicon dioxide based on the total weight of the tablet.

9. The fast water dispersible tablet as claimed in claim 8, wherein the tablet comprises about 1% w/w to about 2% w/w of extragranular colloidal silicon dioxide based on the total weight of the tablet.

10. A fast water dispersible tablet comprising
(i) intragranular components:

| | | |
|---|---|---|
| Capecitabine | 1000 | mg |
| Crospovidone | 30 | mg |
| Colloidal silicon dioxide | 20 | mg |
| Microcrystalline cellulose | 257 | mg |
| Magnesium Stearate | 3.25 | mg |

and
(ii) extragranular components:

| | | |
|---|---|---|
| Colloidal silicon dioxide | 20 | mg |
| Magnesium Stearate | 9.75 | mg |
